SEQUENCE LISTING (88 sequences)

SEQ ID NO: 1 - DNA, 1554 bp, Megasphaera elsdenii
  CDS (1)..(1554)
atgagaaaagtagaaatcattacagctgaacaagcagctcagctcgtaaaagacaacgacacgattacgtctatcggctttgtcagcagcgcccatccggaagcactgaccaaagctttggaaaaacggttcctggacacgaacaccccgcagaacttgacctacatctatgcaggctctcagggtaaacgcgatggccgtgccgctgaacatctggcacacacaggccttttgaaacgcgccatcatcggtcactggcagactgtaccggctatcggtaaactggctgtcgaaaacaagattgaagcttacaacttctcgcagggcacgttggtccactggttccgcgccttggcaggtcataagctcggcgtcttcaccgacatcggtctggaaactttcctcgatccccgtcagctcggcggcaagctcaatgacgtaaccaaagaagacctcgtcaaactgatcgaagtcgatggtcatgaacagcttttctacccgaccttcccggtcaacgtagctttcctccgcggtacgtatgctgatgaatccggcaatatcaccatggacgaagaaatcgggcctttcgaaagcacttccgtagcccaggccgttcacaactgtggcggtaaagtcgtcgtccaggtcaaagacgtcgtcgctcacggcagcctggatccgcgcatggtcaaaatccctggcatctatgtcgactatgttgtcgtagctgctccggaagaccatcagcagacttatgactgcgaatatgatccgtcccttagcggcgaacatcgtgctcctgaaggcgctgctgacgcagctctccccatgagcgctaagaaaatcatcggccgccgcggtgctttggaattgaccgaaaacgctgtcgtcaacctcggcgtcggcgctccggaatacgttgcttccgttgccggtgaagaaggtatcgctgataccattaccttgaccgtcgaaggtggcgctatcggtggtgtaccgcagggcggtgcccgcttcggttcgtcccgtaatgctgatgccatcatcgaccatacttaccagttcgacttctatgatggcggcggtctggacatcgcttacctcggcctggctcagtgcgatggttcgggcaacatcaacgtcagcaagttcggtactaacgttgccggctgtggcggtttccccaacatttcccagcagacaccgaatgtttacttctgcggcaccttcacggctggcggcttgaaaatcgctgtcgaagacggcaaagtcaagatcctccaggaaggcaaagccaagaagttcatcaaagctgtcgaccagatcactttcaacggttcttatgcagcccgcaacggcaaacatgttctctacatcacggaacgctgcgtatttgaactgaccaaagaaggcttgaaactcatcgaagtcgcaccgggcatcgatattgaaaaagatatcctcgctcacatggacttcaagccgatcattgataatccgaaactcatggatgcccgcctcttccaggacggtcccatgggactgaaaaaataa

SEQ ID NO: 2 - protein, 517 aa, Megasphaera elsdenii
MRKVEIITAEQAAQLVKDNDTITSIGFVSSAHPEALTKALEKRFLDTNTPQNLTYIYAGSQGKRDGRAAEHLAHTGLLKRAIIGHWQTVPAIGKLAVENKIEAYNFSQGTLVHWFRALAGHKLGVFTDIGLETFLDPRQLGGKLNDVTKEDLVKLIEVDGHEQLFYPTFPVNVAFLRGTYADESGNITMDEEIGPFESTSVAQAVHNCGGKVVVQVKDVVAHGSLDPRMVKIPGIYVDYVVVAAPEDHQQTYDCEYDPSLSGEHRAPEGAADAALPMSAKKIIGRRGALELTENAVVNLGVGAPEYVASVAGEEGIADTITLTVEGGAIGGVPQGGARFGSSRNADAIIDHTYQFDFYDGGGLDIAYLGLAQCDGSGNINVSKFGTNVAGCGGFPNISQQTPNVYFCGTFTAGGLKIAVEDGKVKILQEGKAKKFIKAVDQITFNGSYAARNGKHVLYITERCVFELTKEGLKLIEVAPGIDIEKDILAHMDFKPIIDNPKLMDARLFQDGPMGLKK

SEQ ID NO: 3 - DNA, 1563 bp, Staphylococcus aureus
  CDS (1)..(1563)
ttgaaacaaatcacatggcacgacttacaacatatcattaaagatggtgatgtgattggtttaccagcattagctgtagccaacttacccgccgaagttctacgtgctgtgttagcgcaacatgacacatatcatacgcccaaagatttaacgtttatattagcgaatgatatccatagtttaggtgccgcaccggatttagatgattttatagaacgtcgcatgattaaacgtgtcattatgagcattttaacggcttcttccaaaacggcacaagcaatgaaaaataatgacattgaagcttattttttaccacaaggtatcattgcaactcattatcgtcagagtaatcaattattacctggagttattactaaaatcggattaaacacagctgttgatcctagatacggtggcggtaaagtaaatacacgaacaactgatgatttagtttcattagtaaccatcaacgatgaaacatacttacattacacattccctagcgttgatgtggcactactgagaggaacatacgcagatcaacaaggtaacatttatttaactcaagaagcgtacttgagcgagtgttatcatgtcgcattaaacgcgaaagccaatcatgggaaagttattgtacaagttaaagctttagttgatggatatcaactaaaaccgaatgaagttgttatcccaggaaatcttgtcgattatgtatacgtcacagaagatgaaaagaatcaccgccaagtaattcagagtcattatttaccagccttgtctggagaagaacgaattgatggaatacctgaacccgcattaccttttaatagtcgcaaattgattctccgacgtgctgctcagtttttaacttatggcgatacaattagcatcggttatggcatcaataatgaactctctaatttattgcacgaagaatgtgttgaacatgatgtgcaaccgattttagatgttggcattttcggtggattcgttgggagtcgtgaacattttggtatgaattacaatgcagatgtgcgcatgcctcatgatcgagcatgggattttatttataacaatggtgtatcagttgcctatcttagctttgctgaggttgatcaatacggcaatgtcaacgtgtcttacttcaatgaccgactaaatggatgtggtggctttatagacattacgcaatctgtaaataaaattatcttttcaggtacttttgtagctggcagtcatgtctcatgccataatcaacgattaaacattgaaactgaaggacaaaaccagaaatttgtatcagatgtgagccatatcgactttaatgcacaatattcacaatcactcgagcaagaagtctattttgttactgagcgtgcagtattcgaactcaccaatcaaggcttgaaactaattgaaattgcaccaggtcttgatttgcataaagatattttgaatcaaatggcttttaaaccaattattgctgatcatttaaaattaattgataccagcatttacaaagaaaaatggggacaacttaaacaatcaattcataaagtatga

SEQ ID NO: 4 - protein, 520 aa, Staphylococcus aureus
LKQITWHDLQHIIKDGDVIGLPALAVANLPAEVLRAVLAQHDTYHTPKDLTFILANDIHSLGAAPDLDDFIERRMIKRVIMSILTASSKTAQAMKNNDIEAYFLPQGIIATHYRQSNQLLPGVITKIGLNTAVDPRYGGGKVNTRTTDDLVSLVTINDETYLHYTFPSVDVALLRGTYADQQGNIYLTQEAYLSECYHVALNAKANHGKVIVQVKALVDGYQLKPNEVVIPGNLVDYVYVTEDEKNHRQVIQSHYLPALSGEERIDGIPEPALPFNSRKLILRRAAQFLTYGDTISIGYGINNELSNLLHEECVEHDVQPILDVGIFGGFVGSREHFGMNYNADVRMPHDRAWDFIYNNGVSVAYLSFAEVDQYGNVNVSYFNDRLNGCGGFIDITQSVNKIIFSGTFVAGSHVSCHNQRLNIETEGQNQKFVSDVSHIDFNAQYSQSLEQEVYFVTERAVFELTNQGLKLIEIAPGLDLHKDILNQMAFKPIIADHLKLIDTSIYKEKWGQLKQSIHKV

SEQ ID NO: 5 - DNA, 1683 bp, Pseudomonas sp. 61-3
  CDS (1)..(1683)
atgagagagaaaccaacgccgggcttgctgcccacacccgcgacgttcatcaacgctcagagtgcgattaccggtctgcgcggccgggatctgttctcgaccctgcgcagcgtggccgcccacggcctgcgtcacccggtgcgcagcgcccgtcatgttctggcactgggcggccagttgggccgcgtgctgctgggcgaaacgctgcacacgccgaacccgaaagacaatcgctttgcggacccgacctggagactgaatccgttttaccggcgcagcctgcaggcctatctgagctggcagaaacaggtcaaaagctggatcgatgaaagcggcatgagtgacgatgaccgcgcccgcgcgcatttcgtcttcgcactgctcaatgacgccgtgtccccctccaataccctgctcaacccgctagcgatcaaggagctgttcaactccggtggcaacagcctggtccgcggtctcagccatttattcgacgacctgatgcacaacaacgggctgcccagtcaggtcaccaaacacgccttcgagattggcaagaccgtggcaaccaccgccgggtccgtggtgtttcgcaacgagctgctcgagctgatgcagtacaagccgatgagcgaaaaacagtacgccaagccgttgctgatcgtcccgccgcagattaacaagtactacattttcgacctcagcccgggtaacagcttcgtccagtacgcattgaagaatggtctgcaggtgttcgtggtcagctggcgtaacccggatgttcgccaccgcgaatggggcctgtccagttacgttgaggcactggaagaagcactgaatgtttgccgcgctatcaccggcgcgcgcgacgtcaatctgatgggcgcctgtgctggcggcctgaccatcgcggctctgcaaggtcatctgcaagccaagcggcaactgcggcgggtctccagcgccagctacctggtcagcctgctggatagccagatagacagcccggcgacgttgttcgccgatgagcagacgctggaagccgccaagcgccattcctatcaacgaggtgtgctcgaggggcgcgacatggcgaaaatcttcgcctggatgcgccccaatgacctgatctggaactactgggtcaacaactacctgctgggcaaagaaccgccggccttcgacattctgtattggaacagtgacaacacgcgcctgccagcggcattccatggcgacctgctggacttcttcaagcacaatccgctgactcaccccggcgggctggaggtctgtggcacgcctatcgatttgcagaaggtcaacgtagacagcttcagcgtggccggcatcaacgaccacatcactccgtgggacgcggtgtaccgctcgaccctgctgctgggtggcgaccggcgcttcgtactgtccaacagcgggcatatccagagcatcctcaacccgccgagcaaccccaagtccaactacatcgagaaccccaagctcagtggcgatccacgcgcctggtattacgacggcacccatgtcgaaggtagctggtggccacgttggctgagctggattcaggagcgctccggtacccaacgcgaaaccctgatggcccttggtaaccagaactatccaccgatggaggcggcgccaggtacctacgtgcgcgtgcgctga

SEQ ID NO: 6 - protein, 560 aa, Pseudomonas sp. 61-3
MREKPTPGLLPTPATFINAQSAITGLRGRDLFSTLRSVAAHGLRHPVRSARHVLALGGQLGRVLLGETLHTPNPKDNRFADPTWRLNPFYRRSLQAYLSWQKQVKSWIDESGMSDDDRARAHFVFALLNDAVSPSNTLLNPLAIKELFNSGGNSLVRGLSHLFDDLMHNNGLPSQVTKHAFEIGKTVATTAGSVVFRNELLELMQYKPMSEKQYAKPLLIVPPQINKYYIFDLSPGNSFVQYALKNGLQVFVVSWRNPDVRHREWGLSSYVEALEEALNVCRAITGARDVNLMGACAGGLTIAALQGHLQAKRQLRRVSSASYLVSLLDSQIDSPATLFADEQTLEAAKRHSYQRGVLEGRDMAKIFAWMRPNDLIWNYWVNNYLLGKEPPAFDILYWNSDNTRLPAAFHGDLLDFFKHNPLTHPGGLEVCGTPIDLQKVNVDSFSVAGINDHITPWDAVYRSTLLLGGDRRFVLSNSGHIQSILNPPSNPKSNYIENPKLSGDPRAWYYDGTHVEGSWWPRWLSWIQERSGTQRETLMALGNQNYPPMEAAPGTYVRVR

SEQ ID NO: 7 - DNA, 1134 bp, Alcanivorax borkumensis SK2
  CDS (1)..(1134)
atgtggatggctaaatcacgattaaaaaaaagtctgcgtgccgttggccacattgttgagcgcaggcgccacccgcaacgctttatccacgtggataaatgcccgtgggaggaagtgtatcgtgacggcatcatggcggtacgccattacagcctaccctctacggctacggctaaaatctcgattaacgatgatttcctgcctgtttcccctgtaaaacaccgcatcccccttttgttggttccggcgctgggtattcattgctggacctacgatttgatgccaaaccgatccatggtccgttatcttatggctcatggttatgaggtctatctggttgactggggaaagccttcagataccgactgcagcctaaatttggacacctacgtcaatcgctggttgccctctgcagttgaaacagtgcgaaaacatgcgcagaccgaaaccatcaacatgatgggctactgcatgggcggactgctgtgcctaatgtatctaggcggccacagtgatgcgccggtgcgtagcctgattaccattgccagccccgtgaattttcacaaaagcggccttttcggcaaggccttagggctggcggctatccctgccatgcagctccatgaccggtttaagattcgtcttgaaccgctcagtgataagctattccatatccctgccagcctcctggcacttggattcaagatgaccaaccctccaggagtggtgcaggcctacatggatctgatccgcaatatcggtgaccgagaatacgtcaccgagtacatgaccatggggcagtggtttaacgacatggtcgattatcctggtgcggtggtgcgtgaggttatcgagaaaatgcttcttgccaatagtctggccaaaggcaaaatccacatcggcggccgcagcgtggatttctcatccattcagcaggatttgctcgcttttgcaggcattaccgacaacattgtcagtcttcgagccgcacgggatatcatccaacttgtcggcagcaaagaaaaacgcttcgaggaagtacctggcggacacgcaggcgctttttgcggttcgaaagcaccttccaatgcctggcgcatcagcgctgactggttggcggcgcgctcagcatag

SEQ ID NO: 8 - protein, 377 aa, Alcanivorax borkumensis SK2
MWMAKSRLKKSLRAVGHIVERRRHPQRFIHVDKCPWEEVYRDGIMAVRHYSLPSTATAKISINDDFLPVSPVKHRIPLLLVPALGIHCWTYDLMPNRSMVRYLMAHGYEVYLVDWGKPSDTDCSLNLDTYVNRWLPSAVETVRKHAQTETINMMGYCMGGLLCLMYLGGHSDAPVRSLITIASPVNFHKSGLFGKALGLAAIPAMQLHDRFKIRLEPLSDKLFHIPASLLALGFKMTNPPGVVQAYMDLIRNIGDREYVTEYMTMGQWFNDMVDYPGAVVREVIEKMLLANSLAKGKIHIGGRSVDFSSIQQDLLAFAGITDNIVSLRAARDIIQLVGSKEKRFEEVPGGHAGAFCGSKAPSNAWRISADWLAARSA

SEQ ID NO: 9 - DNA, 23 bp, Artificial Sequence
  Synthetic DNA
atgagaaaagtagaaatcattac

SEQ ID NO: 10 - DNA, 30 bp, Artificial Sequence
  Synthetic DNA
ttattttttcagtcccatgggaccgtcctg

SEQ ID NO: 11 - DNA, 29 bp, Artificial Sequence
  Synthetic DNA
gtgccatggaacaaatcacatggcacgac

SEQ ID NO: 12 - DNA, 28 bp, Artificial Sequence
  Synthetic DNA
cacgaattcatactttatgaattgattg

SEQ ID NO: 13 - DNA, 40 bp, Artificial Sequence
  Synthetic DNA
gggggccatgggaaaggttcccattattaccgcagatgag

SEQ ID NO: 14 - DNA, 39 bp, Artificial Sequence
  Synthetic DNA
ggggggctcgagtcaggacttcatttccttcagacccat

SEQ ID NO: 15 - DNA, 20 bp, Artificial Sequence
  Synthetic DNA
tcagcgttgcaggatgtagg

SEQ ID NO: 16 - DNA, 23 bp, Artificial Sequence
  Synthetic DNA
tccatgtctgacatgaagtggaa

SEQ ID NO: 17 - DNA, 20 bp, Artificial Sequence
  Synthetic DNA
tgcgccgcagaaaatcaacc

SEQ ID NO: 18 - DNA, 25 bp, Artificial Sequence
  Synthetic DNA
acaagtcaatatggcaaccgaagag

SEQ ID NO: 19 - DNA, 28 bp, Artificial Sequence
  Synthetic DNA
aggagatatacatatggaggcgttcgcc

SEQ ID NO: 20 - DNA, 28 bp, Artificial Sequence
  Synthetic DNA
agatccaactcaggacttctcgcgtacg

SEQ ID NO: 21 - DNA, 20 bp, Artificial Sequence
  Synthetic DNA
tttctcgttcggtcacgatg

SEQ ID NO: 22 - DNA, 22 bp, Artificial Sequence
  Synthetic DNA
tcgctgtttcttaggatgtctc

SEQ ID NO: 23 - DNA, 20 bp, Artificial Sequence
  Synthetic DNA
ccgggctcgatgtttacgac

SEQ ID NO: 24 - DNA, 21 bp, Artificial Sequence
  Synthetic DNA
gacaagtgagtcgcccctatg

SEQ ID NO: 25 - DNA, 20 bp, Artificial Sequence
  Synthetic DNA
ttacgctagggtagaggaag

SEQ ID NO: 26 - DNA, 20 bp, Artificial Sequence
  Synthetic DNA
atggaatcgaatgagcagaa

SEQ ID NO: 27 - DNA, 20 bp, Artificial Sequence
  Synthetic DNA
gacaacgatttgcacgtttc

SEQ ID NO: 28 - DNA, 20 bp, Artificial Sequence
  Synthetic DNA
acgattgctacttccatgtc

SEQ ID NO: 29 - DNA, 20 bp, Artificial Sequence
  Synthetic DNA
atggcttgacgaaggagtgt

SEQ ID NO: 30 - DNA, 23 bp, Artificial Sequence
  Synthetic DNA
gggttttcatccagtcttcttgg

SEQ ID NO: 31 - DNA, 27 bp, Artificial Sequence
  Synthetic DNA
atgagcaataatgcaaacgaccccaca

SEQ ID NO: 32 - DNA, 29 bp, Artificial Sequence
  Synthetic DNA
ggaatcctgctgtccagttattcgttcag

SEQ ID NO: 33 - DNA, 22 bp, Artificial Sequence
  Synthetic DNA
gccgccgaggtactattatgag

SEQ ID NO: 34 - DNA, 20 bp, Artificial Sequence
  Synthetic DNA
aaaggggcgccgaattacag

SEQ ID NO: 35 - DNA, 20 bp, Artificial Sequence
  Synthetic DNA
cgtaagtacgacagtcggtt

SEQ ID NO: 36 - DNA, 20 bp, Artificial Sequence
  Synthetic DNA
gtcatgttctccagcgtctt

SEQ ID NO: 37 - DNA, 29 bp, Artificial Sequence
  Synthetic DNA
atggaatcgacaaataaaacctggacaga

SEQ ID NO: 38 - DNA, 27 bp, Artificial Sequence
  Synthetic DNA
aaaattttcactgtcgttccgatagcc

SEQ ID NO: 39 - DNA, 20 bp, Artificial Sequence
  Synthetic DNA
catttccaggagtcgttgtg

SEQ ID NO: 40 - DNA, 20 bp, Artificial Sequence
  Synthetic DNA
ttgtgcgtaaatccattccc

SEQ ID NO: 41 - DNA, 39 bp, Artificial Sequence
  Synthetic DNA
accagaaaataaaaaatgataaagaaggaaatcgaccaa

SEQ ID NO: 42 - DNA, 19 bp, Artificial Sequence
  Synthetic DNA
ttaattagaacgctcttca

SEQ ID NO: 43 - DNA, 21 bp, Artificial Sequence
  Synthetic DNA
ttgaattgtttcaaaaacgaa

SEQ ID NO: 44 - DNA, 39 bp, Artificial Sequence
  Synthetic DNA
ttggtcgatttccttctttatcattttttattttctggt

SEQ ID NO: 45 - DNA, 20 bp, Artificial Sequence
  Synthetic DNA
aatgttccacaggtacagtc

SEQ ID NO: 46 - DNA, 20 bp, Artificial Sequence
  Synthetic DNA
ccagcctaaggtttaacagg

SEQ ID NO: 47 - DNA, 20 bp, Artificial Sequence
  Synthetic DNA
cacttgaaggacggatcgct

SEQ ID NO: 48 - DNA, 20 bp, Artificial Sequence
  Synthetic DNA
tcgcttaccccttctgcaac

SEQ ID NO: 49 - DNA, 21 bp, Artificial Sequence
  Synthetic DNA
ggcaggatcagcagatggttc

SEQ ID NO: 50 - DNA, 20 bp, Artificial Sequence
  Synthetic DNA
gatgggcacgatcaaaccct

SEQ ID NO: 51 - DNA, 64 bp, Artificial Sequence
  Synthetic DNA
ccggttcgaatctagaaataattttgtttaactttaagaaggagatatacatatgtctgacatg

SEQ ID NO: 52 - DNA, 41 bp, Artificial Sequence
  Synthetic DNA
gaaccaggcggaacctgcagagatccaactcagcgttgcag

SEQ ID NO: 53 - DNA, 64 bp, Artificial Sequence
  Synthetic DNA
ccggttcgaatctagaaataattttgtttaactttaagaaggagatatacatatggcaaccgaa

SEQ ID NO: 54 - DNA, 41 bp, Artificial Sequence
  Synthetic DNA
gaaccaggcggaacctgcagagatccaactcaagccccgcc

SEQ ID NO: 55 - DNA, 57 bp, Artificial Sequence
  Synthetic DNA
tcgaatctagaaataattttgtttaactttaagaaggagatatacatatggaggcgt

SEQ ID NO: 56 - DNA, 32 bp, Artificial Sequence
  Synthetic DNA
ggaacctgcagagatccaactcaggacttctc

SEQ ID NO: 57 - DNA, 57 bp, Artificial Sequence
  Synthetic DNA
tcgaatctagaaataattttgtttaactttaagaaggagatatacatatgtacaaca

SEQ ID NO: 58 - DNA, 31 bp, Artificial Sequence
  Synthetic DNA
ggaacctgcagagatccaactcaggtgcgtt

SEQ ID NO: 59 - DNA, 64 bp, Artificial Sequence
  Synthetic DNA
ccggttcgaatctagaaataattttgtttaactttaagaaggagatatacatatgtttacgaca

SEQ ID NO: 60 - DNA, 41 bp, Artificial Sequence
  Synthetic DNA
gaaccaggcggaacctgcagagatccaactcagatcctaac

SEQ ID NO: 61 - DNA, 64 bp, Artificial Sequence
  Synthetic DNA
ccggttcgaatctagaaataattttgtttaactttaagaaggagatatacatatggccaatcag

SEQ ID NO: 62 - DNA, 37 bp, Artificial Sequence
  Synthetic DNA
caggcggaacctgcagagatccaactcacgtaatcgc

SEQ ID NO: 63 - DNA, 64 bp, Artificial Sequence
  Synthetic DNA
ccggttcgaatctagaaataattttgtttaactttaagaaggagatatacatatggaatcgaat

SEQ ID NO: 64 - DNA, 41 bp, Artificial Sequence
  Synthetic DNA
gaaccaggcggaacctgcagagatccaacctaaatacgctt

SEQ ID NO: 65 - DNA, 64 bp, Artificial Sequence
  Synthetic DNA
ccggttcgaatctagaaataattttgtttaactttaagaaggagatatacatatgcagcagttc

SEQ ID NO: 66 - DNA, 41 bp, Artificial Sequence
  Synthetic DNA
gaaccaggcggaacctgcagagatccaactcattgcaggct

SEQ ID NO: 67 - DNA, 64 bp, Artificial Sequence
  Synthetic DNA
ccggttcgaatctagaaataattttgtttaactttaagaaggagatatacatatgagagagaaa

SEQ ID NO: 68 - DNA, 41 bp, Artificial Sequence
  Synthetic DNA
gaaccaggcggaacctgcagagatccaactcagcgcacgcg

SEQ ID NO: 69 - DNA, 57 bp, Artificial Sequence
  Synthetic DNA
tcgaatctagaaataattttgtttaactttaagaaggagatatacatatgacgtcac

SEQ ID NO: 70 - DNA, 29 bp, Artificial Sequence
  Synthetic DNA
ggaacctgcagagatccaacctagtcgtt

SEQ ID NO: 71 - DNA, 64 bp, Artificial Sequence
  Synthetic DNA
ccggttcgaatctagaaataattttgtttaactttaagaaggagatatacatatgagcaataat

SEQ ID NO: 72 - DNA, 41 bp, Artificial Sequence
  Synthetic DNA
gaaccaggcggaacctgcagagatccaacctatttgatcaa

SEQ ID NO: 73 - DNA, 64 bp, Artificial Sequence
  Synthetic DNA
ccggttcgaatctagaaataattttgtttaactttaagaaggagatatacatatgagtaataca

SEQ ID NO: 74 - DNA, 41 bp, Artificial Sequence
  Synthetic DNA
gaaccaggcggaacctgcagagatccaacttacagttgatc

SEQ ID NO: 75 - DNA, 64 bp, Artificial Sequence
  Synthetic DNA
ccggttcgaatctagaaataattttgtttaactttaagaaggagatatacatatggaatcgaca

SEQ ID NO: 76 - DNA, 41 bp, Artificial Sequence
  Synthetic DNA
gaaccaggcggaacctgcagagatccaactcactgtcgttc

SEQ ID NO: 77 - DNA, 65 bp, Artificial Sequence
  Synthetic DNA
ccggttcgaatctagaaataattttgtttaactttaagaaggagatatacatatgtggatggcta

SEQ ID NO: 78 - DNA, 41 bp, Artificial Sequence
  Synthetic DNA
gaaccaggcggaacctgcagagatccaacctatgctgagcg

SEQ ID NO: 79 - DNA, 64 bp, Artificial Sequence
  Synthetic DNA
ccggttcgaatctagaaataattttgtttaactttaagaaggagatatacatatgaattgtttc

SEQ ID NO: 80 - DNA, 41 bp, Artificial Sequence
  Synthetic DNA
gaaccaggcggaacctgcagagatccaacttaattagaacg

SEQ ID NO: 81 - DNA, 64 bp, Artificial Sequence
  Synthetic DNA
ccggttcgaatctagaaataattttgtttaactttaagaaggagatatacatatgctctccaat

SEQ ID NO: 82 - DNA, 41 bp, Artificial Sequence
  Synthetic DNA
gaaccaggcggaacctgcagagatccaacttaatctgaacg

SEQ ID NO: 83 - DNA, 65 bp, Artificial Sequence
  Synthetic DNA
ccggttcgaatctagaaataattttgtttaactttaagaaggagatatacatatggcggaggcgg

SEQ ID NO: 84 - DNA, 41 bp, Artificial Sequence
  Synthetic DNA
gaaccaggcggaacctgcagagatccaacctaagtgcctgc

SEQ ID NO: 85 - DNA, 64 bp, Artificial Sequence
  Synthetic DNA
ccggttcgaatctagaaataattttgtttaactttaagaaggagatatacatttgatggaactg

SEQ ID NO: 86 - DNA, 41 bp, Artificial Sequence
  Synthetic DNA
gaaccaggcggaacctgcagagatccaactcatcggcgcgc

SEQ ID NO: 87 - DNA, 64 bp, Artificial Sequence
  Synthetic DNA
ccggttcgaatctagaaataattttgtttaactttaagaaggagatatacatatggcgaccggc

SEQ ID NO: 88 - DNA, 41 bp, Artificial Sequence
  Synthetic DNA
gaaccaggcggaacctgcagagatccaactcatgccttggc

(19) United States
(12) Patent Application Publication
(10) Pub. No.: US 2012/0122165 A1
(43) Pub. Date: May 17, 2012

(54) RECOMBINANT MICROORGANISM AND METHOD FOR PRODUCING ALIPHATIC POLYESTER WITH THE USE OF THE SAME

(75) Inventors: Shusei Obata, Nagoya-shi (JP); Masayoshi Muramatsu, Miyoshi-shi (JP); Hiromi Kambe, Seto-shi (JP); Masakazu Ito, Toyota-shi (JP); Takashi Shimamura, Toyota-shi (JP); Katsunori Kohda, Nisshin-shi (JP)

(73) Assignee: TOYOTA JIDOSHA KABUSHIKI KAISHA, Toyota-shi, Aichi (JP)

(21) Appl. No.: 13/387,271

(22) PCT Filed: Jul. 27, 2010
(86) PCT No.: PCT/JP2010/004762

§ 371 Date: Jan. 26, 2012

(30) Foreign Application Priority Data
Jul. 27, 2009 (JP) 2009-174703

(51) Int. Cl. C12P 7/62; C12N 1/21

(52) U.S. Cl. 435/135; 435/252.33

(57) ABSTRACT

This invention provides a recombinant microorganism exhibiting excellent aliphatic polyester productivity and a method for producing an aliphatic polyester with the use of the recombinant microorganism. In this invention, the Megasphaera elsdenii-derived pct gene (and/or the Staphylococcus aureus-derived pct gene) and the Pseudomonas sp. 61-3 strain-derived PHA synthase gene (phaC2 gene) (and/or the Alcanivorax borkumensis SK2 strain-derived PHA synthase gene) are introduced into a host microorganism.

### TECHNICAL FIELD

The present invention relates to a recombinant microorganism to which desired functions have been imparted by introducing a predetermined gene into a host microorganism and a method for producing an aliphatic polyester with the use of the same.

### BACKGROUND ART

Aliphatic polyesters have been gaining attention as biodegradable plastics that can be readily degraded in nature or as “green plastics” that can be synthesized from reproducible carbon resources such as sugar or plant oil. At present, aliphatic polyesters having lactic acid skeletons (e.g., polylactate) have been used in practice.

The technique disclosed in, for example, Patent Document 1 (WO 2006/126796) has been known as a technique of producing an aliphatic polyester such as polylactate with the use of a recombinant microorganism. Patent Document 1 discloses recombinant Escherichia coli obtained by introducing a gene encoding an enzyme that converts lactic acid into lactate CoA and a gene encoding an enzyme that synthesizes polyhydroxyalkanoate with the use of lactate CoA as a substrate into Escherichia coli serving as a host. In the technique disclosed in Patent Document 1, the Clostridium propionicum-derived pct gene is used as a gene encoding an enzyme that converts lactic acid into lactate CoA. In addition, in such case, the Pseudomonas sp. 61-3 strain-derived phaC2 gene is used as a gene encoding an enzyme that synthesizes polyhydroxyalkanoate with the use of lactate CoA as a substrate.

Nevertheless, it cannot be said that a sufficient level of productivity regarding aliphatic polyester such as polylactate is achieved in the case of Patent Document 1. Further, sufficient discussion on the improvement of such productivity has not yet been carried out. For example, Patent Document 2 (WO 2008/062999) discloses an attempt to improve the capacity to synthesize a lactic acid homopolymer or polylactate copolymer with the use of lactate CoA as a substrate by introducing a specific mutation into the Pseudomonas sp. 6-19 strain-derived phaC1 gene.

Meanwhile, the Megasphaera elsdenii-derived gene (propionyl-CoA transferase gene (pct gene)) has been disclosed as a gene encoding an enzyme that converts lactic acid into lactate CoA in, for example, Patent Document 3 (U.S. Pat. No. 7,186,541). However, in Patent Document 3, there is no examination or comparison of the activity levels of a variety of propionyl-CoA transferase genes. In addition, Patent Document 3 does not disclose a technique involving the use of the above gene upon production of aliphatic polyester as disclosed in Patent Document 1.

### CITATION LIST

### Patent Literature

PTL 1: Patent Document 1: WO 2006/126796
PTL 2: Patent Document 2: WO 2008/062999
PTL 3: Patent Document 3: U.S. Pat. No. 7,186,541

### SUMMARY OF INVENTION

### Technical Problem

As described above, the technique of producing an aliphatic polyester such as polylactate with the use of a recombinant microorganism has been problematic in terms of low aliphatic polyester productivity. In addition, it cannot be said that the technique has been sufficiently examined in view of the improvement of productivity. Therefore, it is an object of the present invention to provide a recombinant microorganism that exhibits excellent aliphatic polyester productivity and a method for producing aliphatic polyester with the use of such recombinant microorganism.

### Solution to Problem

As a result of intensive studies in order to achieve the above object, the present inventors have found that a recombinant microorganism into which a predetermined microorganism-derived propionyl-CoA transferase gene and a predetermined microorganism-derived polyhydroxyalkanoate synthase gene have been introduced has significantly excellent productivity regarding aliphatic polyester such as polylactate. This has led to the completion of the present invention.

Specifically, the present invention encompasses the following.

Recombinant microorganisms of the present invention are obtained by introducing a gene selected from among genes (a) to (c) and a gene selected from among genes (d) to (f) shown below into a host microorganism:

(a) a gene that encodes a protein having the amino acid sequence shown in SEQ ID NO: 2 or 4;

(b) a gene that encodes a protein having an amino acid sequence derived from the amino acid sequence shown in SEQ ID NO: 2 or 4 by substitution, deletion, or addition of 1 or more amino acid(s) and having activity of converting lactic acid into lactate CoA;

(c) a gene that hybridizes to a polynucleotide having a nucleotide sequence complementary to the nucleotide sequence shown in SEQ ID NO: 1 or 3 under stringent conditions and encodes a protein having activity of converting lactic acid into lactate CoA;

(d) a gene that encodes a protein having the amino acid sequence shown in SEQ ID NO: 6 or 8;

(e) a gene that encodes a protein having an amino acid sequence derived from the amino acid sequence shown in SEQ ID NO: 6 or 8 by substitution, deletion, or addition of 1 or more amino acid(s) and having activity of synthesizing polylactate with the use of lactate CoA as a substrate; and

(f) a gene that hybridizes to a polynucleotide having a nucleotide sequence complementary to the nucleotide sequence shown in SEQ ID NO: 5 or 7 under stringent conditions and encodes a protein having activity synthesizing polylactate with the use of lactate CoA as a substrate.

Particularly preferably, the recombinant microorganism of the present invention is obtained with the use of Escherichia coli as a host microorganism. In addition, the recombinant microorganism of the present invention may be a microorganism into which not only the two above genes but also a gene encoding an enzyme involved in the aliphatic polyester synthesis system have been introduced. A recombinant microorganism into which the two above genes have been introduced can synthesize a polylactate homopolymer with the use of a carbon source in a medium. A recombinant microorganism into which not only the two above genes but also a gene encoding an enzyme involved in the aliphatic polyester synthesis system have been introduced can synthesize lactic acid copolymer with the use of a carbon source in a medium. Herein, the term “lactic acid copolymer” refers to a polymer having a polymer skeleton comprising a lactic acid skeleton and a non-lactic-acid hydroxyalkanoate skeleton.

In addition, according to the present invention, the aforementioned method for producing an aliphatic polyester with the use of the recombinant microorganism of the present invention can be provided. Specifically, the method for producing an aliphatic polyester of the present invention comprises the steps of culturing a microorganism and collecting an aliphatic polyester from a medium.

### Advantageous Effects of Invention

According to the present invention, a recombinant microorganism having an excellent aliphatic polyester-producing capacity can be provided. Specifically, the recombinant microorganism of the present invention has significantly excellent aliphatic polyester-producing capacity compared with a conventional recombinant microorganism. With the use of the recombinant microorganism of the present invention, a method for producing an aliphatic polyester with excellent productivity can be provided.

### BRIEF DESCRIPTION OF DRAWINGS

FIG. 1 is a characteristic diagram showing the evaluation results regarding the activity of converting lactic acid into lactate CoA for the Clostridium propionicum-derived propionyl-CoA transferase gene, the Megasphaera elsdenii-derived propionyl-CoA transferase gene, and the Staphylococcus aureus-derived propionyl-CoA transferase gene.

FIG. 2 is a characteristic diagram showing the evaluation results regarding polylactate productivity for a variety of polyhydroxyalkanoate synthase genes expressed with the Megasphaera elsdenii-derived propionyl-CoA transferase gene.

### DESCRIPTION OF EMBODIMENTS

Hereinafter, the recombinant microorganism and the method for producing an aliphatic polyester using the same of the present invention are described in detail.

The recombinant microorganism of the present invention is obtained by introducing a predetermined propionyl-CoA transferase gene (pct gene) and a predetermined polyhydroxyalkanoate synthase gene into a host microorganism.

Propionyl-CoA Transferase Gene

In the present invention, examples of propionyl-CoA transferase genes (hereinafter referred to as pct genes(s)) include a Megasphaera elsdenii-derived gene and a Staphylococcus aureus-derived gene. SEQ ID NO: 1 shows the nucleotide sequence of the coding region of the Megasphaera elsdenii-derived pct gene. SEQ ID NO: 2 shows the amino acid sequence of a protein encoded by such pct gene. In addition, SEQ ID NO: 3 shows the nucleotide sequence of the coding region of the Staphylococcus aureus-derived pct gene. SEQ ID NO: 4 shows the amino acid sequence of a protein encoded by such pct gene. A protein having the amino acid sequence shown in SEQ ID NO: 2 or 4 has propionyl-CoA transferase activity, and particularly activity of synthesizing lactate CoA with the use of lactic acid as a substrate.

In addition, examples of a pct gene of the present invention are not limited to a gene having the nucleotide sequence encoding the amino acid sequence shown in SEQ ID NO: 2 or 4 and may include a gene encoding a protein having an amino acid sequence derived from the amino acid sequence by deletion, substitution, or addition of 1 or more amino acid(s) and having activity of converting lactic acid into lactate CoA. Herein, the term “1 or more amino acid(s)” refers to, for example, 1 to 20, preferably 1 to 10, more preferably 1 to 7, further preferably 1 to 5, and particularly preferably 1 to 3 amino acids.

Further, a pct gene of the present invention may be a gene that encodes a protein having an amino acid sequence with a sequence similarity of, for example, 70% or higher, preferably 80% or higher, more preferably 90% or higher, and most preferably 95% or higher with the amino acid sequence shown in SEQ ID NO: 2 or 4 and having activity of converting lactic acid into lactate CoA. Herein, the sequence similarity value refers to the value obtained in the default setting with the use of a computer program implemented with the BLAST algorithm and a database containing gene sequence information.

Furthermore, a pct gene of the present invention may be a gene which has a polynucleotide that hybridizes to at least a portion of a gene having the nucleotide sequence shown in SEQ ID NO: 1 or 3 under stringent conditions and encodes a protein having activity of converting lactic acid into lactate CoA. Herein, the term “under stringent conditions” refers to what are called conditions that cause formation of a specific hybrid but not a non-specific hybrid. For instance, conditions of hybridization with 6×SSC (sodium chloride/sodium citrate) at 45 degrees C. and subsequent washing with 0.2 to 1×SSC and 0.1% SDS at 50 degrees C. to 65 degrees C. can be referred to. Alternatively, conditions of hybridization with 1×SSC at 65 degrees C. to 70 degrees C. and subsequent washing with 0.3×SSC at 65 degrees C. to 70 degrees C. can be referred to as such conditions. Hybridization can be carried out by a conventionally known method such as the method described in J. Sambrook et al. Molecular Cloning, A Laboratory Manual, 2nd Ed., Cold Spring Harbor Laboratory (1989).

In addition, deletion, substitution, or addition of amino acid(s) can be carried out by a technique known in the art by modifying a nucleotide sequence encoding a transcription factor described above. Mutagenesis in a nucleotide sequence can be caused by a known method such as the Kunkel method, the gapped duplex method, or a method similar to such a known method. For instance, mutagenesis can be caused with the use of a mutagenesis kit (e.g., Mutant-K or Mutant-G (product name, TAKARA Bio)) based on a site-directed mutagenesis method, an LA PCR in vitro Mutagenesis series kit (product name, TAKARA Bio), or the like. Alternatively, a mutagenesis method may be a method using a chemical mutagen represented by EMS (ethyl methanesulfonate), 5-bromouracil, 2-aminopurine, hydroxylamine, N-methyl-N′-nitro-N-nitrosoguanidine, or a different carcinogenic compound, a method comprising radiation treatment using radioactive rays such as X-rays, alpha-rays, beta-rays, gamma-rays, or an ion beam, or a method comprising ultraviolet treatment.

Polyhydroxyalkanoate Synthase Gene

Polyhydroxyalkanoate synthase genes (also referred to as PHA synthase genes) are known to exist in many microorganisms, as disclosed in Patent Document 1 (WO 2006/126796). Particularly in the present invention, a specific polyhydroxyalkanoate synthase gene is expressed in a host microorganism with a pct gene described above. Specifically, as a polyhydroxyalkanoate synthase gene used in the present invention, the Pseudomonas sp. 61-3 strain-derived polyhydroxyalkanoate synthase gene (phaC2 gene) and/or the Alcanivorax borkumensis SK2 strain-derived polyhydroxyalkanoate synthase gene can be used. SEQ ID NO: 5 shows the nucleotide sequence of the coding region of the Pseudomonas sp. 61-3 strain-derived polyhydroxyalkanoate synthase gene (phaC2 gene). SEQ ID NO: 6 shows the amino acid sequence of a protein encoded by the phaC2 gene. In addition, SEQ ID NO: 7 shows the nucleotide sequence of the coding region of the Alcanivorax borkumensis SK2 strain-derived polyhydroxyalkanoate synthase gene. SEQ ID NO: 8 shows the amino acid sequence of a protein encoded by the polyhydroxyalkanoate synthase gene. A protein having the amino acid sequence shown in SEQ ID NO: 6 or 8 has polyhydroxyalkanoate-synthesizing activity, and particularly activity of synthesizing polylactate with the use of lactate CoA as a substrate or activity of synthesizing a polylactate-based copolymer with the use of lactate CoA and a different hydroxyalkanoate as a substrate.

In addition, examples of a PHA synthase gene of the present invention are not limited to a gene having the nucleotide sequence encoding the amino acid sequence shown in SEQ ID NO: 6 or 8 and may include a gene encoding a protein having an amino acid sequence derived from the amino acid sequence by deletion, substitution, or addition of 1 or more amino acid(s) and having activity of synthesizing polylactate with the use of lactate CoA as a substrate. Herein, the term “1 or more amino acid(s)” refers to, for example, 1 to 20, preferably 1 to 10, more preferably 1 to 7, and further preferably 1 to 5, and particularly preferably 1 to 3 amino acid(s).

Further, a PHA synthase gene of the present invention may be a gene that encode a protein having an amino acid sequence with a sequence similarity of, for example, 70% or higher, preferably 80% or higher, more preferably 90% or higher, and most preferably 95% or higher with the amino acid sequence shown in SEQ ID NO: 6 or 8 and having activity of synthesizing polylactate with the use of lactate CoA as a substrate. Herein, the sequence similarity value refers to the value obtained in the default setting with the use of a computer program implemented with the BLAST algorithm and a database containing gene sequence information.

Furthermore, a PHA synthase gene of the present invention may be a gene which has a polynucleotide that hybridizes to at least a portion of a gene having the nucleotide sequence shown in SEQ ID NO: 5 or 7 under stringent conditions and encodes a protein having activity of synthesizing polylactate with the use of lactate CoA as a substrate. In addition, the term “stringent conditions” refers to the same as defined in the paragraph describing “the propionyl-CoA transferase gene”.

Also, techniques exemplified in the paragraph describing “the propionyl-CoA transferase gene” can be used for amino acid deletion, substitution, or addition.

Host Microorganisms

Examples of a host microorganisms of the present invention include: bacteria belonging to the genus Pseudomonas, such as the Pseudomonas sp. 61-3 strain; bacteria belonging to the genus Ralstonia, such as R. eutropha; bacteria belonging to the genus Bacillus, such as Bacillus subtilis; bacteria belonging to the genus Escherichia, such as Escherichia coli; bacteria belonging to the genus Corynebacterium; yeasts belonging to the genus Saccharomyces, such as Saccharomyces cerevisiae; and yeasts belonging to the genus Candida, such as Candida maltosa. It is particularly preferable to use Escherichia coli as a host microorganism.

A vector used for introduction of the aforementioned genes into a host cell can be a vector capable of autonomously replicating in a host, which is preferably in the form of plasmid DNA or phage DNA. Examples of a vector to be introduced into Escherichia coli include: plasmid DNAs such as pBR322, pUC18, and pBLuescript II; and phage DNAs such as EMBL3, M13, and lambda-gtII. Examples of a vector to be introduced into a yeast include YEp13 and YCp50.

Insertion of both or either of the aforementioned genes into a vector can be carried out using a gene recombinant technique known by those in the art. In addition, upon recombination, it is preferable to ligate the gene(s) downstream of a promoter capable of regulating transcription. Any promoter can be used as long as it can regulate gene transcription in a host. For instance, in a case in which Escherichia coli is used as a host, a trp promoter, a lac promoter, a PL promoter, a PR promoter, a T7 promoter, or the like can be used. In a case in which a yeast is used as a host, a gal1 promoter, a gal10 promoter, or the like can be used.

Further, a terminator sequence, an enhancer sequence, a splicing signal sequence, a poly-A addition signal sequence, a ribosome binding sequence (SD sequence), a selection marker gene, and the like, which can be used in a microorganism used for gene introduction, can be ligated to a vector according to need. Examples of a selection marker gene include a gene involved in intracellular biosynthesis of a nutrient such as an amino acid or a nucleic acid and a gene encoding a fluorescent protein such as luciferase, in addition to drug-resistant genes such as an ampicillin-resistant gene, a tetracycline-resistant gene, a neomycin-resistant gene, a kanamycin-resistant gene, and a chloramphenicol-resistant gene.

The above vector can be introduced into a microorganism by a method known by those in the art. Examples of a method for introducing a vector into a microorganism include a calcium phosphate method, an electroporation method, a spheroplast method, a lithium acetate method, a conjugal transfer method, and a method using calcium ions.

Production of Aliphatic Polyester

An aliphatic polyester of interest can be produced by culturing a recombinant microorganism obtained by introducing a pct gene and a PHA synthase gene described above into a host microorganism in a medium containing a carbon source so as to cause generation and accumulation of aliphatic polyester in culture bacterial cells or a culture, followed by collection of aliphatic polyester from the culture bacterial cells or the culture. The above recombinant microorganism synthesizes lactic acid from a sugar in a sugar metabolism pathway and then propionyl-CoA transferase encoded by the pct gene converts lactic acid into lactate CoA. In addition, in the recombinant microorganism, PHA synthase encoded by the PHA synthase gene synthesizes an aliphatic polyester comprising lactic acid as a building block with the use of lactate CoA as a substrate. Herein, an aliphatic polyester may be polylactate (homopolymer) comprising a building block consisting of lactic acid or a lactic acid-based copolymer comprising a building block consisting of lactic acid and non-lactic-acid hydroxyalkanoate. When polylactate (homopolymer) is synthesized, non-lactic-acid hydroxyalkanoate is not added to a medium, or a host microorganism is caused to lack a non-lactic-acid hydroxyalkanoate biosynthesis pathway. Meanwhile, when a lactic acid-based copolymer comprising a building block consisting of lactic acid and non-lactic-acid hydroxyalkanoate is synthesized, non-lactic-acid hydroxyalkanoate can be added to a medium, or a host microorganism is allowed to have a non-lactic-acid hydroxyalkanoate biosynthesis pathway.

Examples of carbon sources include carbohydrates such as glucose, fructose, sucrose, and maltose. In addition, a fat-and-oil-related substance with a carbon number of 4 or higher can be used as a carbon source. Examples of a fat-and-oil-related substance with a carbon number of 4 or higher include: natural fat and oil such as corn oil, soybean oil, safflower oil, sunflower oil, olive oil, coconut oil, palm oil, rapeseed oil, fish oil, whole oil, pig oil, or bovine oil; fatty acid such as butanoic acid, pentanoic acid, hexanoic acid, octanoic acid, decanoic acid, lauric acid, oleic acid, palmitic acid, linolenic acid, linoleic acid, myristic acid, or a fatty acid ester thereof; and alcohol such as octanol, lauryl alcohol, oleyl alcohol, palmityl alcohol, or an ester thereof.

Examples of nitrogen sources include peptone, meat extract, yeast extract, and corn steep liquor, in addition to ammonium salts such as ammonia, ammonium chloride, ammonium sulfate, and ammonium phosphate. Examples of inorganic matter include potassium phosphate monobasic, potassium phosphate dibasic, magnesium phosphate, magnesium sulfate, and sodium chloride.

In general, it is preferable to carry out culture under aerobic conditions for shaking culture or the like at 25 degrees C. to 37 degrees C. for at least 24 hours after the initiation of the expression of the pct gene and the PHA synthase gene. It is possible to add an antibiotic such as kanamycin, ampicillin, or tetracycline to a medium during culture. In a case in which introduction of either or both of the pct gene and the PHA synthase gene has been carried out under the control of induction promoters, it is preferable to carry out culture for at least 24 hours after the addition of factors that induce transcription from the promoters to a medium.

It is particularly preferable to culture recombinant Escherichia coli into which the pct gene and the PHA synthase gene described above have been introduced so as to produce polylactate. In such method, polylactate can be produced without adding monomer components that constitute a polymer of interest such as lactic acid to a medium, which is advantageous in terms of production cost.

In addition, an aliphatic polyester such as lactic acid can be collected by a method known to those in the art. For example, harvest from a culture solution via centrifugation and washing are carried out, followed by drying. Then, dried bacterial cells are suspended in chloroform and heated. Thus, a polyester of interest is extracted with the chloroform fraction. Further, methanol is added to the chloroform solution for deposition of the polyester and then the supernatant is removed via filtration or centrifugation, followed by drying. Accordingly, a purified polyester can be obtained. Confirmation regarding a collected polyester as polylactate can be carried out by a general method such as gas chromatography or a nuclear magnetic resonance method.

### EXAMPLES

The present invention is hereafter described in greater detail with reference to the following examples, although the technical scope of the present invention is not limited thereto.

### Example 1

### Evaluation of a Variety of pct Genes

In this Example, the Clostridium propionicum-derived pct gene, the Megasphaera elsdenii-derived pct gene, and the Staphylococcus aureus-derived pct gene were evaluated regarding activity of converting lactic acid into lactate CoA. A pTV118N-C.P PCT vector, a pTV118N-M.E PCT vector, and a pTV118N-S.A PCT vector were prepared for introduction of the Clostridium propionicum-derived pct gene, the Megasphaera elsdenii-derived pct gene, and the Staphylococcus aureus-derived pct gene, respectively.

First, the genomes of M. elsdenii (ATCC17753), S. aureus (ATCC10832), and C. propionicum (ATCC25522) were obtained by a general method. Each pct gene was obtained by a PCR method. The following primers were used for amplification of a DNA fragment containing the M. elsdenii-derived pct gene: MePCTN: 5′-atgagaaaagtagaaatcattac-3′ (SEQ ID NO: 9); and MePCTC: 5′-ttattttttcagtcccatgggaccgtcctg-3′ (SEQ ID NO: 10). The following primers were used for amplification of a DNA fragment containing the S. aureus (ATCC10832)-derived pct gene: SpctN: 5′-gtgccatggaacaaatcacatggcacgac-3′ (SEQ ID NO: 11); and SpctC: 5′-cacgaattcatactttatgaattgattg-3′ (SEQ ID NO: 12). The following primers were used for amplification of a DNA fragment containing the C. propionicum (ATCC25522)-derived pct gene: CpPCTN: 5′-gggggccatgggaaaggttcccattattaccgcagatgag-3′ (SEQ ID NO: 13); and CpPCTC: 5′-ggggggctcgagtcaggacttcatttccttcagacccat-3′ (SEQ ID NO: 14). In addition, information registered with NCBI was used as a reference for primer nucleotide sequences. However, for M. elsdemnii, the sequence described in WO02/42418 was referred to.

Each gene was amplified from the relevant genome under the following conditions: PCR (enzyme KOD plus) (94 degrees C. for 1 min)×1, (94 degrees C. for 0.5 min, 50 degrees C. for 0.5 min, 72 degrees C. for 2 min)×30, (94 degrees C. for 2 min). Amplified fragments were separately introduced into a TOPO BluntII vector, followed by sequencing. As a result, C. propionicum-derived pct and S. aureus-derived pct were found to be completely identical to AJ276553 and MW0211, respectively, which are the sequences registered in the NCBI database. The nucleotide sequence of M. elsdenii-derived pct had 97.8% homology to the previously reported sequence. However, a single-site difference was found in the amino acid sequence thereof.

The above pct genes from C. propionicum, M. elsdenii, and S. aureus obtained by PCR were inserted into NcoI-BamHI, EcoR1-PstI, and NcoI-EcoRI sites of a pTV118N vector (Takara Bio Inc.), respectively. Thus, expression plasmids (pTV118N-C.P PCT, pTV118N-M.E PCT, and pTV118N-S.A PCT) were produced. Thereafter, these expression plasmids were separately introduced into Escherichia coli W3110.

The obtained transformed Escherichia coli was precultured, followed by inoculation at 2% in a 200-ml LB/2 L flask and culture at 37 degrees C. and 180 rpm for 3 h. Expression induction was carried out at approximately OD₆₀₀=0.5 with the use of 10 mM IPTG, followed by culture at 30 degrees C. and 80 rpm for 6 h. Next, bacterial cells were collected via centrifugation and cultured at 37 degrees C. with M9 (+1.5% Glucose, 10 mM MgSO₄, 10 mM calcium pantothenate) (OD=20, 3 ml). Sampling was carried out in an adequate manner.

The amount of synthesized lactate CoA was determined for comparison of C. propionicum-, M. elsdenii-, and S. aureus-derived pct genes regarding activity of converting lactic acid into lactate CoA. First, bacterial cells were collected (1×10⁵ cells) for preparation of samples (n=3). Samples were applied to a suction filter system and washed twice with Milli Q water. A filter (turned upside-down) was placed in a petri dish containing an MeOH solution (2 ml) and allowed to stand at room temperature for 10 minutes. A portion of the MeOH solution (1.6 ml) was transferred into a centrifuge tube and mixed with chloroform (1.6 ml) and Milli Q water (640 ul), followed by suspension. After centrifugation at 4600 g and 4 degrees C. for 5 min, the water+MeOH layer (1.5 ml) was subjected to centrifugal filtration with a 5 k ultra-filtration membrane (Millopore) for approximately 2 h. The filtrate was collected and lyophilized. The resultant was concentrated 200-fold and dissolved with Milli Q water containing a secondary internal standard substance, followed by CE-MS analysis. “Pressure-Assisted Capillary Electrophoresis Electrospray Ionization Mass Spectrometry for Analysis of Multivalent Anions” (Anal. Chem 2002, 74, 6224-6229) was referred to for CE-MS analysis conditions.

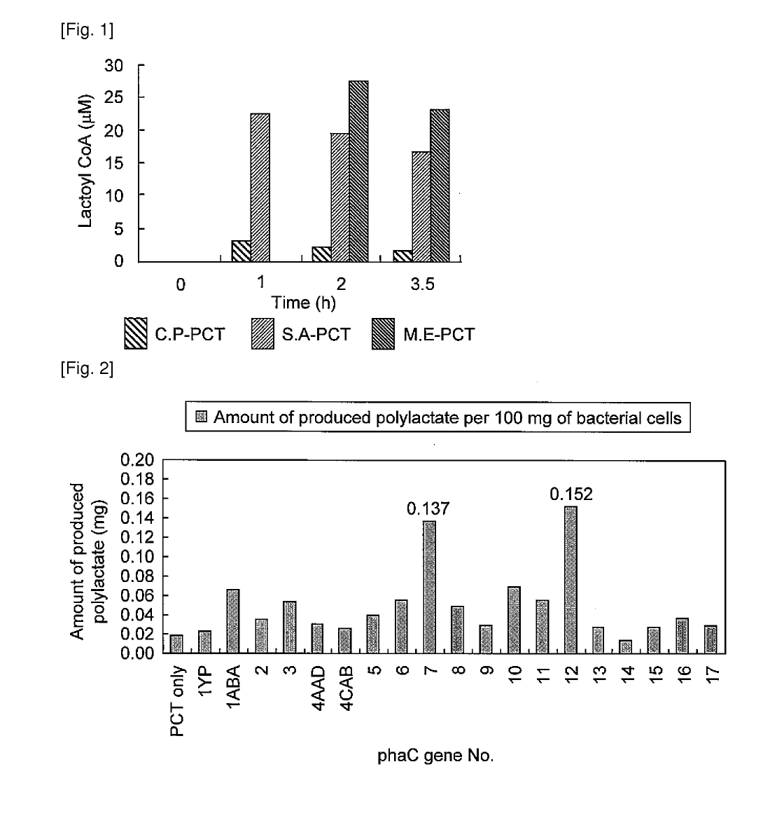

The results are shown in FIG. 1. As shown in FIG. 1, it was revealed that the Megasphaera elsdenii-derived pct gene and the Staphylococcus aureus-derived pct gene have activity of converting lactic acid into lactate CoA at a level significantly higher than that of the Clostridium propionicum-derived pct gene. The results revealed that a substrate used for reaction of synthesizing an aliphatic polyester with a basic skeleton partially comprising or consisting of lactic acid can be sufficiently supplied with the use of the Megasphaera elsdenii-derived pct gene and/or the Staphylococcus aureus-derived pct gene.

### Example 2

### Evaluation of a Variety of PHA Synthase Genes

In this Example, a variety of PHA synthase genes were evaluated regarding the polylactate productivity in a case in which the genes were allowed to be expressed with the Megasphaera elsdenii-derived pct gene that had been evaluated as having significantly high activity of converting lactic acid into lactate CoA in Example 1. Table 1 lists PHA synthase genes examined in this Example. In table 1, for Rhodobacter sphaeroides (No. 1) and Rhodospirillum rubrum (No. 4), since a plurality of genes registered with different accession numbers have been found, such plurality of genes were examined.

TABLE 1

| | | | | Depository | |
|---|---|---|---|---|---|
| N^{o} | Strain | Accession No. | Class | organization | N^{o} |
| 1 | Rhodobacter sphaeroides | YP354337 | I | ATCC | BAA-808D |
| | | ABA79557 | I | | |
| 2 | Azorhizobium caulinodans | | I | NBRC | 14845 |
| 3 | Rhizobium etli CFN 42 | | I | ″ | 15573 |
| 4 | Rhodospirillum rubrum | AAD53179 | I | ATCC | 25903 |
| | | CAB65395 | I | | |
| 5 | Colwellia psychrerythraea 34H | | I | ″ | BAA-681D |
| 6 | Chromobacterium violaceum | | I | ″ | 12472D |
| 7 | Pseudomonas sp. 61-3 | | II | JCM | 10015 |
| 8 | Hyphomonas neptunium | | II | NBRC | 14232 |
| 9 | Haloquadratum walsbyi | | III | JCM | 12895 |
| 10 | Haloarcula marismortui | | III | ″ | 8966 |
| 11 | Synechocystis sp. PCC6803 | | III | ATCC | 27184D |
| 12 | Alcanivorax borkumensis SK2 | | III | ″ | |
| 13 | Bacillus cereus | | IV | ″ | 14579D |
| 14 | Acinetobacter baumannii ATCC 17978 | | — | ″ | 17978 |
| 15 | Magnetospirillum magneticum AMB-1 | | — | ATCC | 700264 |
| 16 | Xanthomonas campestris pv. Campestris | | — | ″ | 33913D |
| 17 | Ralstonia eutropha H16 | | I | | |

In addition, in table 1, “Class I” refers to a PHA synthase gene having high activity while having high substrate specificity, “Class II” refers to a PHA synthase gene having low substrate specificity while having low activity, “Class III” refers to a PHA synthase gene for which the presence of phaE is required in a PHA synthase reaction, and “Class IV” refers to a PHA synthase gene for which the presence of phaR is required in a PHA synthase reaction.

DNA fragments containing 19 types of PHA synthase genes from 17 types of microorganisms shown in Nos. 1 to 17 were amplified by a single instance of PCR or two instances of PCR. The DNA fragments were introduced into pTV118N vectors into which the Megasphaera elsdenii-derived pct gene had been introduced. Tables 2 and 3 list 1st PCR primers designed for amplification of DNA fragments.

TABLE 2

| | | phaC gene | | |
|---|---|---|---|---|
| | | registration | | |
| No | Strain name | name | Primer name | Sequence |
| 1 | Rhodobacter | R.sphae-YP | RsphaeroidesF | TCAGCGTTGCAGGATGTAGG |
| | sphaeroides | | | (SEQ ID NO: 15) |
| | | | RsphaeroidesR | TCCATGTCTGACATGAAGTGGAA |
| | | | | (SEQ ID NO: 16) |
| | | R.sphae-ABA | Rhodobacter-fwd 2 | TGCGCCGCAGAAAATCAACC |
| | | | | (SEQ ID NO: 17) |
| | | | Rhodobacter-rvs 2 | ACAAGTCAATATGGCAACCGAAGAG |
| | | | | (SEQ ID NO: 18) |
| 2 | Azorhizobium | A.cauli | Azorhizobium-fwd 3 | AGGAGATATACATATGGAGGCGTTCGCC |
| | caulinodans | | | (SEQ ID NO: 19) |
| | | | Azorhizobium-rvs 3 | AGATCCAACTCAGGACTTCTCGCGTACG |
| | | | | (SEQ ID NO: 20) |
| 3 | Rhizobium etli | R. etil | Rhizobium-fwd 2 | TTTCTCGTTCGGTCACGATG |
| | CFN 42 | | | (SEQ ID NO: 21) |
| | | | Rhizobium-rvs 2 | TCGCTGTTTCTTAGGATGTCTC |
| | | | | (SEQ ID NO: 22) |
| 4 | Rhodospirillum | R.rubru-AAD | R.rubrumF | CCGGGCTCGATGTTTACGAC |
| | rubrum | | | (SEQ ID NO: 23) |
| | | R.rubru-CAB | R.rubrumR | GACAAGTGAGTCGCCCCTATG |
| | | | | (SEQ ID NO: 24) |
| 5 | Colwellia | C. psych | ColwelliaF | TTACGCTAGGGTAGAGGAAG |
| | psychrerythraea | | | (SEQ ID NO: 25) |
| | 34H | | ColwelliaR | ATGGAATCGAATGAGCAGAA |
| | | | | (SEQ ID NO: 26) |
| 6 | Chromobacterium | C. viola | C.violaceumF | GACAACGATTTGCACGTTTC |
| | violaceum | | | (SEQ ID NO: 27) |
| | | | C.violaceumR | ACGATTGCTACTTCCATGTC |
| | | | | (SEQ ID NO: 28) |
| 7 | Pseudomonas sp. | Ps61-3.C2 | P. sp.61-3 (phaC2)- | ATGGCTTGACGAAGGAGTGT |
| | 61-3 | | fwd 2 | (SEQ ID NO: 29) |
| | | | P. sp.61-3 (phaC2)- | GGGTTTTCATCCAGTCTTCTTGG |
| | | | rvs 2 | (SEQ ID NO: 30) |
| 8 | Hyphomonas | H.neptu | | |
| | neptunium | | | |
| 9 | Haloquadratum | H.walsb | HwalsbphaEC1stFwd | ATGAGCAATAATGCAAACGACCCCACA |
| | walsbyi | | | (SEQ ID NO: 31) |
| | | | HwalsbphaEC1stRvs | GGAATCCTGCTGTCCAGTTATTCGTTCAG |
| | | | | (SEQ ID NO: 32) |
| 10 | Haloarcula | H.maris | HmarisphaEC1stFwd | GCCGCCGAGGTACTATTATGAG |
| | marismortui | | | (SEQ ID NO: 33) |
| | | | HmarisphaEC1stRvs | AAAGGGGCGCCGAATTACAG |
| | | | | (SEQ ID NO: 34) |
| | | | HaloarculaPhaEF | CGTAAGTACGACAGTCGGTT |
| | | | | (SEQ ID NO: 35) |
| | | | HaloarculaPhaER | GTCATGTTCTCCAGCGTCTT |
| | | | | (SEQ ID NO: 36) |

TABLE 3

| | | phaC gene | | |
|---|---|---|---|---|
| | | registration | | |
| No | Strain name | name | Primer name | Sequence |
| 11 | Synechocystis | S.sp. | SynecphaEC1stFwd | ATGGAATCGACAAATAAAACCTGGACAGA |
| | sp. PCC6803 | | | (SEQ ID NO: 37) |
| | | | SynecphaEC1stRvs | AAAATTTTCACTGTCGTTCCGATAGCC |
| | | | | (SEQ ID NO: 38) |
| 12 | Alcanivorax | A.borku-YP | A.borkumensisF | CATTTCCAGGAGTCGTTGTG (SEQ ID NO: 39) |
| | borkumensis | | A.borkumensisR | TTGTGCGTAAATCCATTCCC (SEQ ID NO: 40) |
| | SK2 | | | |
| 13 | Bacillus cereus | B.cereus | BcereusphaC1stFwd | ACCAGAAAATAAAAAATGATAAAGAAGGAAATCGA |
| | | | | CCAA (SEQ ID NO: 41) |
| | | | BcereusphaC1stRvs | TTAATTAGAACGCTCTTCA (SEQ ID NO: 42) |
| | | | BcereusphaR1stFwd | TTGAATTGTTTCAAAAACGAA (SEQ ID NO: 43) |
| | | | BcereusphaR1stRvs | TTGGTCGATTTCCTTCTTTATCATTTTTTATTTTC |
| | | | | TGGT (SEQ ID NO: 44) |
| 14 | Acinetobacter | A.bauma | A.baumanniiF | AATGTTCCACAGGTACAGTC (SEQ ID NO: 45) |
| | baumannii | | A.baumanniiR | CCAGCCTAAGGTTTAACAGG (SEQ ID NO: 46) |
| 15 | Magnetospirillum | M.magne-BAE | M.magneticumF | CACTTGAAGGACGGATCGCT (SEQ ID NO: 47) |
| | magneticum | | M.magneticumR | TCGCTTACCCCTTCTGCAAC (SEQ ID NO: 48) |
| 16 | Xanthomonas | X.campe | X.campestrisF | GGCAGGATCAGCAGATGGTTC (SEQ ID NO: 49) |
| | campestris | | X.campestrisR | GATGGGCACGATCAAACCCT (SEQ ID NO: 50) |
| | pv. Campestris | | | |
| 17 | Ralstonia | R.eutro | | |
| | eutropha H16 | | | |

Tables 4 and 5 list 2nd PCR primers designed for amplification of DNA fragments.

TABLE 4

| | | phaC gene | | |
|---|---|---|---|---|
| | | registration | | |
| No | Strain name | name | Primer name | Sequence |
| 1 | Rhodobacter | R.sphae-YP | RYP3543372ndFwd | CCGGTTCGAATCTAGAAATAATTTTGTTTAACTTTAAGAAG |
| | sphaeroides | | | GAGATATACATATGTCTGACATG (SEQ ID NO: 51) |
| | | | RYP3543372ndRev | GAACCAGGCGGAACCTGCAGAGATCCAACTCAGCGTTGCAG |
| | | | | (SEQ ID NO: 52) |
| | | R.sphae-ABA | RABA795572ndFwd | CCGGTTCGAATCTAGAAATAATTTTGTTTAACTTTAAGAAG |
| | | | | GAGATATACATATGGCAACCGAA (SEQ ID NO: 53) |
| | | | RABA795572ndRev | GAACCAGGCGGAACCTGCAGAGATCCAACTCAAGCCCCGCC |
| | | | | (SEQ ID NO: 54) |
| 2 | Azorhizobium | A.cauli | Azorhizo-fwd | TCGAATCTAGAAATAATTTTGTTTAACTTTAAGAAGGAGAT |
| | caulinodans | | | ATACATATGGAGGCGT (SEQ ID NO: 55) |
| | | | Azorhizo-rvs | GGAACCTGCAGAGATCCAACTCAGGACTTCTC |
| | | | | (SEQ ID NO: 56) |
| 3 | Rhizobium etli | R.etil | Rhizo-fwd | TCGAATCTAGAAATAATTTTGTTTAACTTTAAGAAGGAGAT |
| | CFN 42 | | | ATACATATGTACAACA (SEQ ID NO: 57) |
| | | | Rhizo-rvs | GGAACCTGCAGAGATCCAACTCAGGTGCGTT |
| | | | | (SEQ ID NO: 58) |
| 4 | Rhodospirillum | R.rubru-AAD | RrubruAAD2ndFwd | CCGGTTCGAATCTAGAAATAATTTTGTTTAACTTTAAGAAG |
| | rubrum | | | GAGATATACATATGTTTACGACA (SEQ ID NO: 59) |
| | | | RrubruAAD2ndRvs | GAACCAGGCGGAACCTGCAGAGATCCAACTCAGATCCTAAC |
| | | | | (SEQ ID NO: 60) |
| | | R.rubru-CAB | Rhodospirillum-fwd | CCGGTTCGAATCTAGAAATAATTTTGTTTAACTTTAAGAAG |
| | | | | GAGATATACATATGGCCAATCA(SEQ ID NO: 61) |
| | | | Rhodospirillum-rvs | CAGGCGGAACCTGCAGAGATCCAACTCACGTAATCGC |
| | | | | (SEQ ID NO: 62) |
| 5 | Colwellia | C.psych | Colwellia2ndFwd | CCGGTTCGAATCTAGAAATAATTTTGTTTAACTTTAAGAAG |
| | psychrerythraea | | | GAGATATACATATGGAATCGAAT (SEQ ID NO: 63) |
| | 34H | | Colwellia2ndRev | GAACCAGGCGGAACCTGCAGAGATCCAACCTAAATACGCTT |
| | | | | (SEQ ID NO: 64) |

TABLE 5

| | | phaC gene | | |
|---|---|---|---|---|
| | | registration | | |
| No | Strain name | name | Primer name | Sequence |
| 6 | Chromobacterium | C.viola | CviolaphaC2ndFwd | CCGGTTCGAATCTAGAAATAATTTTGTTTAACTTTAAGAAG |
| | violaceum | | | GAGATATACATATGCAGCAGTTC (SEQ ID NO: 65) |
| | | | CviolaphaC2ndRvs | GAACCAGGCGGAACCTGCAGAGATCCAACTCATTGCAGGCT |
| | | | | (SEQ ID NO: 66) |
| 7 | Pseudomonas | Ps61-3.C2 | PspC22ndFwd | CCGGTTCGAATCTAGAAATAATTTTGTTTAACTTTAAGAAG |
| | sp. | | | GAGATATACATATGAGAGAGAAA (SEQ ID NO: 67) |
| | 61-3 | | PspC22ndRvs | GAACCAGGCGGAACCTGCAGAGATCCAACTCAGCGCACGCG |
| | (phaC2) | | | (SEQ ID NO: 68) |
| 8 | Hyphomonas | H.neptu | Hypho-fwd | TCGAATCTAGAAATAATTTTGTTTAACTTTAAGAAGGAGAT |
| | neptunium | | | ATACATATGACGTCACn (SEQ ID NO: 69) |
| | | | Hypho-rvs | GGAACCTGCAGAGATCCAACCTAGTCGTT |
| | | | | (SEQ ID NO: 70) |
| 9 | Haloquadratum | H.walsb | HwalsbphaEC2ndFwd | CCGGTTCGAATCTAGAAATAATTTTGTTTAACTTTAAGAAG |
| | walsbyi | | | GAGATATACATATGAGCAATAAT (SEQ ID NO: 71) |
| | | | HwalsbphaEC2ndRvs | GAACCAGGCGGAACCTGCAGAGATCCAACCTATTTGATCAA |
| | | | | (SEQ ID NO: 72) |
| 10 | Haloarcula | H.maris | HmarisphaEC2ndFwd | CCGGTTCGAATCTAGAAATAATTTTGTTTAACTTTAAGAAG |
| | marismortui | | | GAGATATACATATGAGTAATACA (SEQ ID NO: 73) |
| | | | HmarisphaEC2ndRvs | GAACCAGGCGGAACCTGCAGAGATCCAACTTACAGTTGATC |
| | | | | (SEQ ID NO: 74) |
| 11 | Synechocystis | S.sp. | SynecphaEC2ndFwd | CCGGTTCGAATCTAGAAATAATTTTGTTTAACTTTAAGAAG |
| | sp. | | | GAGATATACATATGGAATCGACA (SEQ ID NO: 75) |
| | PCC6803 | | SynecphaEC2ndRvs | GAACCAGGCGGAACCTGCAGAGATCCAACTCACTGTCGTTC |
| | | | | (SEQ ID NO: 76) |
| 12 | Alcanivorax | A.borku-YP | Aborku2ndFwd | CCGGTTCGAATCTAGAAATAATTTTGTTTAACTTTAAGAAG |
| | borkumensis | | | GAGATATACATATGTGGATGGCTA (SEQ ID NO: 77) |
| | SK2 | | Aborku2ndRvs | GAACCAGGCGGAACCTGCAGAGATCCAACCTATGCTGAGCG |
| | | | | (SEQ ID NO: 78) |
| 13 | Bacillus cereus | B.cereus | BcereusphaRC2ndFwd | CCGGTTCGAATCTAGAAATAATTTTGTTTAACTTTAAGAAG |
| | | | | GAGATATACATATGAATTGTTTC (SEQ ID NO: 79) |
| | | | BcereusphaRC2ndRvs | GAACCAGGCGGAACCTGCAGAGATCCAACTTAATTAGAACG |
| | | | | (SEQ ID NO: 80) |
| 14 | Acinetobacter | A.bauma | Abauma2ndFwd | CCGGTTCGAATCTAGAAATAATTTTGTTTAACTTTAAGAAG |
| | baumannii | | | GAGATATACATATGCTCTCCAAT (SEQ ID NO: 81) |
| | | | Abauma2ndRvs | GAACCAGGCGGAACCTGCAGAGATCCAACTTAATCTGAACG |
| | | | | (SEQ ID NO: 82) |
| 15 | Magnetospirillum | M.magne-BAE | Mmagne2ndFwd | CCGGTTCGAATCTAGAAATAATTTTGTTTAACTTTAAGAAG |
| | magneticum | | | GAGATATACATATGGCGGAGGCGG (SEQ ID NO: 83) |
| | | | Mmagne2ndRvs | GAACCAGGCGGAACCTGCAGAGATCCAACCTAAGTGCCTGC |
| | | | | (SEQ ID NO: 84) |
| 16 | Xanthomonas | X.campe | Xanthomonas2ndFwd | CCGGTTCGAATCTAGAAATAATTTTGTTTAACTTTAAGAAG |
| | campestris | | | GAGATATACATTTGATGGAACTG (SEQ ID NO: 85) |
| | pv. Campestris | | Xanthomonas2ndRev | GAACCAGGCGGAACCTGCAGAGATCCAACTCATCGGCGCGC |
| | | | | (SEQ ID NO: 86) |
| 17 | Ralstonia | R.eutro | Reutro2ndfwd | CCGGTTCGAATCTAGAAATAATTTTGTTTAACTTTAAGAAG |
| | eutropha H16 | | | GAGATATACATATGGCGACCGGC (SEQ ID NO: 87) |
| | | | Reutro2ndrvs | GAACCAGGCGGAACCTGCAGAGATCCAACTCATGCCTTGGC |
| | | | | (SEQ ID NO: 88) |

In addition, tables 6 and 7 list reaction conditions for PCR with the use of the above primers.

TABLE 6

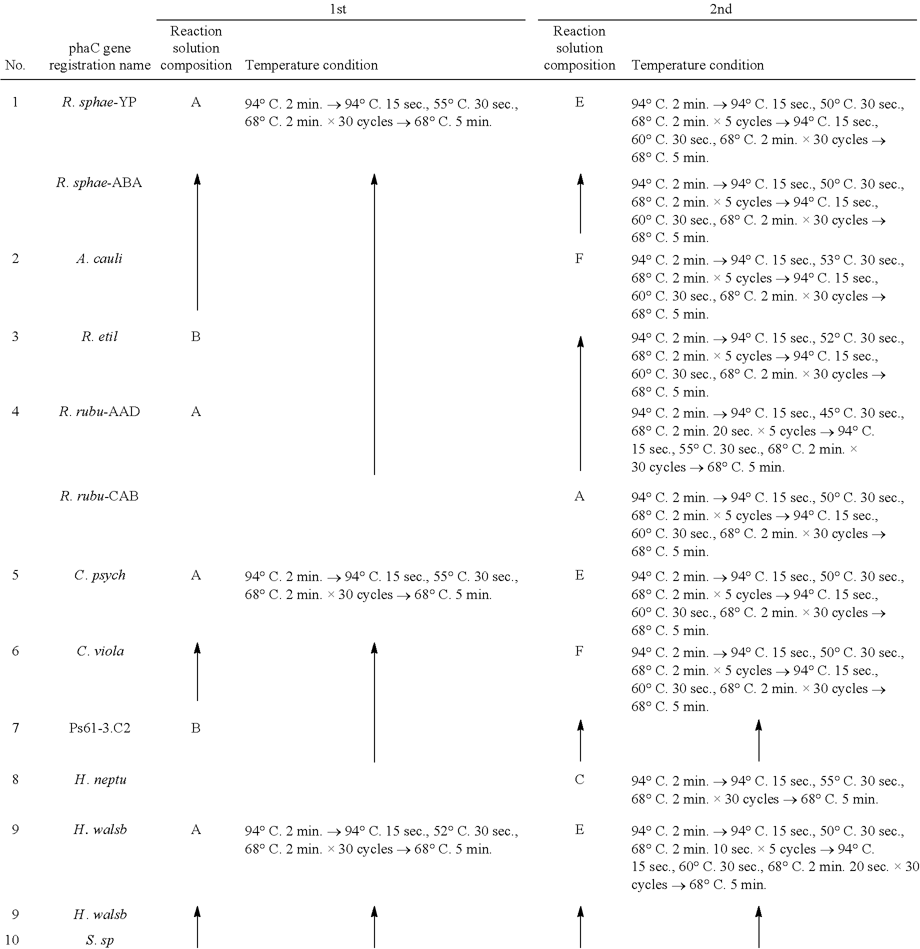

TABLE 7

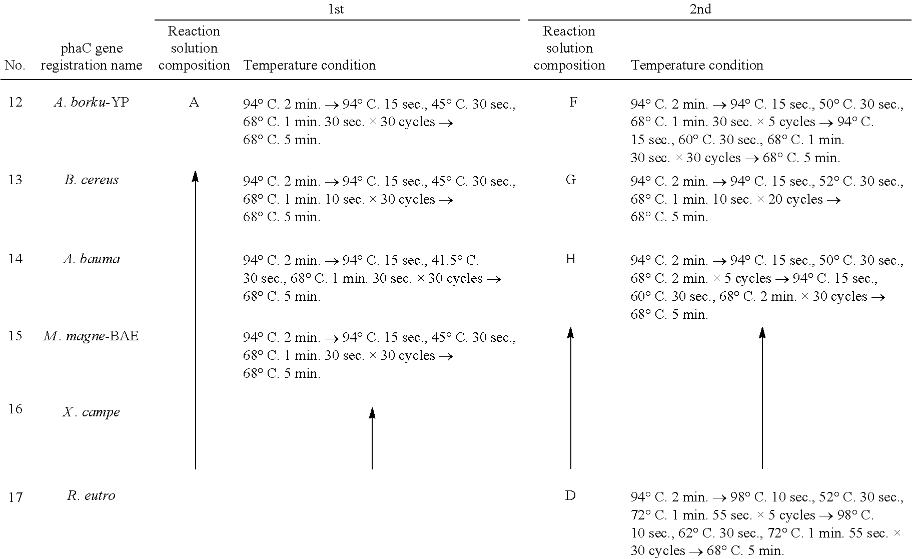

In addition, table 8 lists reaction solution compositions A to H for reaction conditions listed in tables 6 and 7.

TABLE 8

| Reaction solution composition A | Reaction solution composition B |
|---|---|
| 5 μl 10× Buffer for KOD-Plus Ver. 2 (final 1×) | 5 μl 10× Buffer for KOD -Plus Ver. 2 (final 1×) |
| 5 μl 2.5 mM dNTPs (final 0.25 mM each) | 5 μl 2 mM dNTPs (final 0.2 mM each) |
| 2 μl 25 mM MgSO4 (final 1.5 mM) | 2 μl 25 mM MgSO4 (final 1.5 mM) |
| 1.5 μl PrimerF(10 pmol/μ) (final 0.3 μM) | 1.5 μl PrimerF(10 pmol/μ) (final 0.3 μM) |
| 1.5 μl PrimerR(10 pmol/μ) (final 0.3 μM) | 1.5 μl PrimerR(10 pmol/μ) (final 0.3 μM) |
| 10~200 ng templateDNA genome | 10~200 ng templateDNA genome |
| 1 μl KOD-Plus(1 U/μl) (final 1 U/50 μl) | 1 μl KOD -P/lus-(1 U/μl) (final 1 U/50 μl) |
| sterile deionaized water up to 50 μl | sterile deionaized water up to 50 μl |
| Reaction solution composition C | Reaction solution composition D |
| 5 μl 10× Buffer for KOD -Plus Ver. 2 (final 1×) | 5 μl 10× Pyrobest Buffer II (final 1×) |
| 5 μl 2 mM dNTPs (final 0.2 mM each) | 5 μl 2.5 mM dNTPs (final 0.25 mM each) |
| 2 μl 25 mM MgSO4 (final 1.5 mM) | 1.5 μl PrimerF(10 pmol/μ) (final 0.3 μM) |
| 2 μl PrimerF(10 pmol/μ) (final 0.3 μM) | 1.5 μl PrimerR(10 pmol/μ) (final 0.3 μM) |
| 2 μl PrimerR(10 pmol/μ) (final 0.3 μM) | 37 μg template eutropha/pet plasmid |
| 10~200 ng templateDNA genome | 1 μl Pyrobest(U/μl) (final U/50 μl) |
| 1 μl KOD-Plus(1 U/μl) (final 1 U/50 μl) | sterile deionaized water up to 50 μl |
| sterile deionaized water up to 50 μl | |
| Reaction solution composition E | Reaction solution composition F |
| 5 μl 10× Buffer for KOD-Plus Ver. 2 (final 1×) | 5 μl 10× Buffer for KOD-Plus Ver. 2 (final 1×) |
| 5 μl 2.5 mM dNTPs (final 0.25 mM each) | 5 μl 2.5 mM dNTPs (final 0.25 mM each) |
| 2 μl 25 mM MgSO4 (final 1.5 mM) | 2 μl 25 mM MgSO4 (final 1.5 mM) |
| 1.5 μl PrimerF(10 pmol/μ) (final 0.3 μM) | 1.5 μl PrimerF(10 pmol/μ) (final 0.3 μM) |
| 1.5 μl PrimerR(10 pmol/μ) (final 0.3 μM) | 1.5 μl PrimerR(10 pmol/μ) (final 0.3 μM) |
| 1 μl templateDNA(1stPCRproduct, diluted | 1 μl templateDNA(1stPCRproduct, diluted |
| 1/500 after purification) | 1/1000 after purification) |
| 1 μl KOD-Plus(1 U/μl) (final 1 U/50 μl) | 1 μl KOD-Plus(1 U/μl) (final 1 U/50 μl) |
| sterile deionaized water up to 50 μl | sterile deionaized water up to 50 μl |
| Reaction solution composition G | |
| (No addition of primers) | Reaction solution composition H |
| 5 μl 10× Buffer for KOD-Plus Ver. 2 (final 1×) | 5 μl 10× Buffer for KOD-Plus Ver. 2 (final 1×) |
| 5 μl 2.5 mM dNTPs (final 0.25 mM each) | 5 μl 2.5 mM dNTPs (final 0.25 mM each) |
| 2 μl 25 mM MgSO4 (final 1.5 mM) | 2 μl 25 mM MgSO4 (final 1.5 mM) |
| 1 μl templateDNA(phaR 1stPCRproduct, | 1.5 μl PrimerF(10 pmol/μ) (final 0.3 μM) |
| purified without dilution) | 1.5 μl PrimerR(10 pmol/μ) (final 0.3 μM) |
| 1 μl KOD-Plus(1 U/μl)(final 1 U/50 μl) | 1 μl PCR reaction solution described above |
| sterile deionaized water up to 50 μl | (unpurified) |
| | 1 μl KOD-Plus(1 U/μl) (final 1 U/50 μl) |
| | sterile deionaized water up to 50 μl |
| Reaction solution composition G′ | |
| 5 μl 10× Buffer for KOD-Plus Ver. 2 (final 1×) | |
| 5 μl 2.5 mM dNTPs (final 0.25 mM each) | |
| 2 μl 25 mM MgSO4 (final 1.5 mM) | |
| 1.5 μl PrimerF(10 pmol/μ) (final 0.3 μM) | |
| 1.5 μl PrimerR(10 pmol/μ) (final 0.3 μM) | |
| 1 μl PCR reaction solution described above | |
| (unpurified) | |
| 1 μl KOD-Plus(1 U/μl) (final 1 U/50 μl) | |
| sterile deionaized water up to 50 μl | |

In addition, in the case of the pha gene shown in No. 13, two genes (phaR and phaC) were found to exist with another gene sandwiched therebetween. Therefore, after separate cloning by 1st PCR, they were ligated to each other to result in a single gene by 2nd PCR. Further, PCR (reaction solution composition: G′; temperature conditions: 94 degrees C. for 2 minutes, followed by “94 degrees C. for 15 seconds, 50 degrees C. for 30 seconds, 68 degrees C. for 1 minute 40 seconds”×5 cycles, followed by “94 degrees C. for 15 seconds, 60 degrees C. for 30 seconds, 68 degrees C. for 1 minute 40 seconds”×30 cycles, followed by 68 degrees C. for 5 minutes) was again carried out for ligation with a vector.
In addition, regarding the phaC genes shown in Nos. 2, 3, and 8, a purified 2nd PCR product and a pTV118N-PCT-C1 vector were separately digested with restriction enzymes (XbaI and PstI (Takara Bio Inc.)). Each resultant and a 10× loading buffer (Takara Bio Inc.) were loaded on agarose gel (0.8%, TAE), followed by separation via electrophoresis, cleavage, and purification. Purification was carried out with the use of a MinElute Gel Extraction Kit (QIAGEN) in accordance with the relevant protocol. Ligation and transformation were carried out with the use of a Ligation-Convenience Kit (Nippon Gene Co., Ltd.) and ECOS competent E. coli JM109 (Nippon Gene Co., Ltd.), respectively, in accordance with the relevant protocol. The obtained transformant was cultured in an LB-Amp medium (2 ml), followed by plasmid extraction with the use of a QIAprep Spin Miniprep Kit (QIAGEN). A sequence reaction was carried out with the use of a Big Dye Terminator v3.1 Cycle Sequencing Kit (Applied Biosystems). Sequences were confirmed with the use of a DNA sequencer 3100 Genetic Analyzer (Applied Biosystems).

Further, regarding the phaC genes shown in Nos. 1, 4 to 7, and 9 to 17, ligation was carried out with the use of an In-Fusion 2.0 Dry-Down PCR Cloning Kit (Clontech Laboratories) for the convenience of experimental operations or in view of the presence of a PstI site in the phaC gene (Nos. 4, 6, 10, and 12). The other sites were treated in the manner described above.

A variety of phaC genes obtained above were separately incorporated into pTV118N-M.E PCT such that the relevant vectors were obtained. Each obtained vector was introduced into an Escherichia coli W3110 competent cell such that a recombinant Escherichia coli capable of expressing any of the Megasphaera elsdenii-derived pct gene and the PHA synthase genes described above was prepared. The thus obtained each recombinant Escherichia coli was inoculated in an LB medium containing ampicillin, followed by overnight static culture at 37 degrees C. The obtained colony was inoculated in an LB liquid medium containing ampicillin (2 mL), followed by shaking culture in a test tube at 37 degrees C. to result in OD₆₀₀=0.6 to 1.0. The resultant was designated as a preculture solution.

Next, the preculture solution (2 mL) was added to an M9 medium (200 mL) containing ampicillin, 2% glucose, and 0.1 mM IPTG, followed by rotary culture with the use of a 500-mL baffled Erlenmeyer flask at 30 degrees C. and 130 rpm for 48 hours.

After the termination of culture, the culture solution was transferred to a 50-mL Corning tube, followed by harvest under conditions of 3000 rpm for 15 minutes. The supernatant was discarded. Thereafter, the resultant was stored overnight in a freezer at −80 degrees C. so as to become frozen. Thereafter, lyophilization was carried out for 2 days with the use of a lyophilizer. Then, dried bacterial cells (100 mg) were transferred to a pressure-resistant reaction tube. Chloroform (1.6 mL) was added thereto. Further, a mixed solution of methanol and sulfuric acid (methanol:sulfuric acid=17:3 (volume ratio)) (1.6 mL) was added thereto, followed by reflux in a water bath set to 95 degrees C. for 3 hours. Subsequently, the pressure-resistant reaction tube was removed and cooled to room temperature. The solution contained therein was transferred to a test tube. Ultrapure water (0.8 mL) was added to the test tube, followed by mixing with a vortex mixer. The resultant was allowed to stand still. After the resultant had been allowed to stand still for a sufficient time period, the chloroform phase (the lower phase) was collected in a certain amount with a Pasteur pipette. The chloroform phase was filtered via an organic-solvent-resistant filter (0.2 um mesh) and transferred to a GC-MS vial bottle. Thus, an analysis sample was prepared.

HP6890/5973 (Hewlett-Packard) was used as a GC-MS apparatus. BD-1 122-1063 (internal diameter: 0.25 mm; length: 60 m; membrane thickness: 1 um; Agilent Technologies) was used as a column. Temperature increase conditions were as follows: retention at 120 degrees C. for 5 minutes, temperature increase to 200 degrees C. at a rate of 10 degrees C./min, temperature increase to 300 degrees C. at a rate of 20 degrees C./min, and retention for 8 minutes.

FIG. 2 shows the results of comparison of recombinant Escherichia coli in terms of polylactate productivity. As shown in FIG. 2, the recombinant Escherichia coli obtained with the use of the Pseudomonas sp. 61-3 strain-derived PHA synthase gene (phaC2 gene) shown in No. 7 and the Alcanivorax borkumensis SK2 strain-derived PHA synthase gene showed a significantly higher level of polylactate productivity than that of recombinant Escherichia coli obtained with the use of a different microorganism-derived PHA synthase gene. The above results revealed that polylactate productivity is significantly improved via coexpression of the Megasphaera elsdenii-derived pct gene and the Pseudomonas sp. 61-3 strain-derived PHA synthase gene (phaC2 gene) or the Alcanivorax borkumensis SK2 strain-derived PHA synthase gene. In addition, in this Example, lactic acid was not separately added to a medium, and thus polylactate was synthesized in a usual medium containing a carbon source (glucose). Further, the Alcanivorax borkumensis SK2 strain-derived PHA synthase gene does not exhibit the relevant activity by itself, suggesting that it is a gene for which the presence of phaE is required in a PHA synthesis reaction (Class III shown in table 1). However, although no phaE gene was introduced in this Example, it was revealed that the Alcanivorax borkumensis SK2 strain-derived PHA synthase gene can exhibit PHA synthase activity by itself.

1. A recombinant microorganism obtained by introducing a gene selected from among genes (a) to (c) and a gene selected from among genes (d) to (f) shown below into a host microorganism:
(a) a gene that encodes a protein having the amino acid sequence shown in SEQ ID NO: 2 or 4;
(b) a gene that encodes a protein having an amino acid sequence with a sequence similarity of 70% or higher with the amino acid sequence shown in SEQ ID NO: 2 or 4 and having activity of converting lactic acid into lactate CoA;
(c) a gene that hybridizes to a polynucleotide having a nucleotide sequence complementary to the nucleotide sequence shown in SEQ ID NO: 1 or 3 under stringent conditions and encodes a protein having activity of converting lactic acid into lactate CoA;
(d) a gene that encodes a protein having the amino acid sequence shown in SEQ ID NO: 6 or 8;
(e) a gene that encodes a protein having an amino acid sequence with a sequence similarity of 70% or higher with the amino acid sequence shown in SEQ ID NO: 6 or 8 and having activity of synthesizing polylactate with the use of lactate CoA as a substrate; and
(f) a gene that hybridizes to a polynucleotide having a nucleotide sequence complementary to the nucleotide sequence shown in SEQ ID NO: 5 or 7 under stringent conditions and encodes a protein having activity synthesizing polylactate with the use of lactate CoA as a substrate.

2. The recombinant microorganism according to claim 1, wherein the host microorganism is Escherichia coli.

3. A method for producing an aliphatic polyester, comprising culturing the recombinant microorganism according to claim 1 in a medium and collecting an aliphatic polyester.

4. The method for producing an aliphatic polyester according to claim 3, wherein the aliphatic polyester to be collected is an aliphatic polyester having a lactic acid skeleton.

5. The method for producing an aliphatic polyester according to claim 3, wherein the aliphatic polyester to be collected is polylactate.

6. The method for producing an aliphatic polyester according to claim 3, wherein lactic acid is not added to the medium when the recombinant microorganism is cultured.
